(19) United States
(12) Patent Application Publication
(10) Pub. No.: US 2023/0123436 A1
(43) Pub. Date: Apr. 20, 2023

(54) ENDOSCOPE

(71) Applicant: FUJIFILM Corporation, Tokyo (JP)

(72) Inventors: Makito Komukai, Kanagawa (JP); Hiroyuki Iwane, Kanagawa (JP)

(73) Assignee: FUJIFILM Corporation, Tokyo (JP)

(21) Appl. No.: 18/067,730

(22) Filed: Dec. 19, 2022

Related U.S. Application Data
(63) Continuation of application No. PCT/JP2021/027442, filed on Jul. 26, 2021.

(30) Foreign Application Priority Data
Jul. 30, 2020 (JP) 2020-129458

(51) Int. Cl. A61B 1/00; A61B 1/12

(52) U.S. Cl. CPC A61B 1/00091; A61B 1/126

(57) ABSTRACT

The endoscope includes: an insertion part having a distal end surface provided on a distal end side to be inserted into a subject; an observation window that is provided in the distal end surface and that is used to observe an inside of the subject; a fluid jetting nozzle that is provided in the distal end surface and that jets a fluid toward the observation window; and a forceps port that is provided in the distal end surface and that is used to lead out a treatment tool or to suck the fluid. The distal end surface has water repellency in a nozzle facing region provided between the fluid jetting nozzle and the observation window, and has hydrophilicity in a forceps port adjacent region provided on a side opposite to the nozzle facing region with the forceps port interposed therebetween.

### CROSS-REFERENCE TO RELATED APPLICATIONS

The present application is a Continuation of PCT International Application No. PCT/JP2021/027442 filed on Jul. 26, 2021 claiming priority under 35 U.S.C § 119(a) to Japanese Patent Application No. 2020-129458 filed on Jul. 30, 2020. Each of the above applications is hereby expressly incorporated by reference, in its entirety, into the present application.

### BACKGROUND OF THE INVENTION

### 1. Field of the Invention

The present invention relates to an endoscope, and particularly relates to an endoscope for improving a liquid removability of an observation window disposed on a distal end surface of an insertion part.

### 2. Description of the Related Art

An observation window for capturing subject light from a site to be observed and an illumination window for irradiating the site to be observed with illumination light are disposed on a distal end surface of an insertion part of an endoscope. In addition, a fluid jetting nozzle that jets a cleaning liquid (for example, water) and a gas (for example, air) toward the observation window is disposed on the distal end surface in order to remove an attachment such as a body fluid adhering to the observation window.

At a time of cleaning the observation window, first, the cleaning liquid is jetted from a jet port of the fluid jetting nozzle to remove the attachment adhering to the observation window, and then a gas is jetted from the jet port to remove the cleaning liquid remaining on the observation window.

JP2016-202707A discloses an endoscope that enables a fluid jetted from a jet port to flow to an observation window and to an adjacent region to the observation window in cleaning the observation window. In this endoscope, a fluid guide portion, a first fluid route that guides a part of the fluid guided by the fluid guide portion to the observation window, and a second fluid route that guides a fluid deviated from the fluid guide portion to the above-described adjacent region are provided between a fluid jetting nozzle and the observation window.

JP2016-22006A discloses an endoscope capable of removing a residual liquid remaining on an observation window by supplying air from an air/liquid supply nozzle. In this endoscope, at least a part of a flat portion of a distal end cover, a window surface of the observation window, and at least a part of an inclined portion are formed by a distal end cover with a high affinity to a liquid as surface characteristics.

### SUMMARY OF THE INVENTION

A forceps port for leading out a treatment tool or sucking the fluid is provided on the distal end surface of the endoscope. In a vicinity of an inside of an opening of the forceps port, liquids, such as cleaning water for the observation window or cleaning water for an observation target site, may not be completely sucked and may remain. There is a problem in that, in a case where a gas is jetted from the jet port of the fluid jetting nozzle in a state in which the liquid remains near the opening of the forceps port, the liquid (water droplets) may remain in the observation window, or the liquid may be reflected in an observation image because the liquid remaining near the opening of the forceps port is continuously moved in a direction of the observation window by the jetted gas.

The present invention has been made in view of such circumstances, and an object of the present invention is to provide an endoscope capable of improving a liquid removability of an observation window.

In order to achieve the object of the present invention, according to the present invention, there is provided an endoscope comprising: an insertion part having a distal end surface provided on a distal end side to be inserted into a subject; an observation window that is provided in the distal end surface and that is used to observe an inside of the subject; a nozzle that is provided in the distal end surface and that jets a fluid toward the observation window; and a forceps port that is provided in the distal end surface and that is used to lead out a treatment tool or to suck the fluid, in which the distal end surface has water repellency in a nozzle facing region provided between the nozzle and the observation window, and has hydrophilicity in a forceps port adjacent region provided on a side opposite to the nozzle facing region with the forceps port interposed therebetween.

In one aspect of the present invention, it is preferable that the distal end surface has a forceps port forming surface portion in which the forceps port is formed, and a protruding surface portion formed so as to protrude toward a front side, which is the distal end side, with respect to the forceps port forming surface portion, and at least one of the observation window or the nozzle is disposed on the protruding surface portion.

In one aspect of the present invention, it is preferable that the protruding surface portion has an observation window-disposed surface portion in which the observation window is disposed, and a nozzle-disposed surface portion in which the nozzle is disposed, and the observation window-disposed surface portion protrudes to the front side with respect to the nozzle-disposed surface portion.

In one aspect of the present invention, it is preferable that the protruding surface portion has water repellency.

In one aspect of the present invention, it is preferable that the forceps port is disposed at a position close to the nozzle facing region.

In one aspect of the present invention, it is preferable that the distal end surface has water repellency in a nozzle peripheral region provided on a side opposite to the forceps port with the nozzle facing region interposed therebetween.

In one aspect of the present invention, it is preferable that the nozzle has a jet port for jetting the fluid, and at least a part of the forceps port is provided on an observation window side with respect to a reference line, which is an extension line of the jet port.

In one aspect of the present invention, it is preferable that the distal end surface has hydrophilicity in a fluid discharge region provided on a side opposite to the nozzle facing region with the observation window interposed therebetween.

According to the present invention, it is possible to improve a liquid removability of an observation window of an endoscope.

### BRIEF DESCRIPTION OF THE DRAWINGS

FIG. 1 is an overall view of an endoscope according to the present embodiment.

FIG. 2 is a perspective view showing a configuration of a distal end surface of an insertion part of the endoscope shown in FIG. 1.

FIG. 3 is a front view of the distal end surface shown in FIG. 2.

FIG. 4 is a cross-sectional view taken along line Iv-Iv in FIG. 2.

FIG. 5 is a plan view showing a configuration of a fluid guide portion and a fluid route of a fluid.

FIG. 6 is a view showing a water-repellent region and a hydrophilic region of the distal end surface.

FIG. 7 is a view illustrating a flow of a liquid on a distal end surface of an endoscope of a comparative example.

FIG. 8 is a view illustrating a flow of a liquid on the distal end surface of the endoscope of the present embodiment.

### DESCRIPTION OF THE PREFERRED EMBODIMENTS

Hereinafter, preferred embodiments of an endoscope according to the present invention will be described with reference to the accompanying drawings.

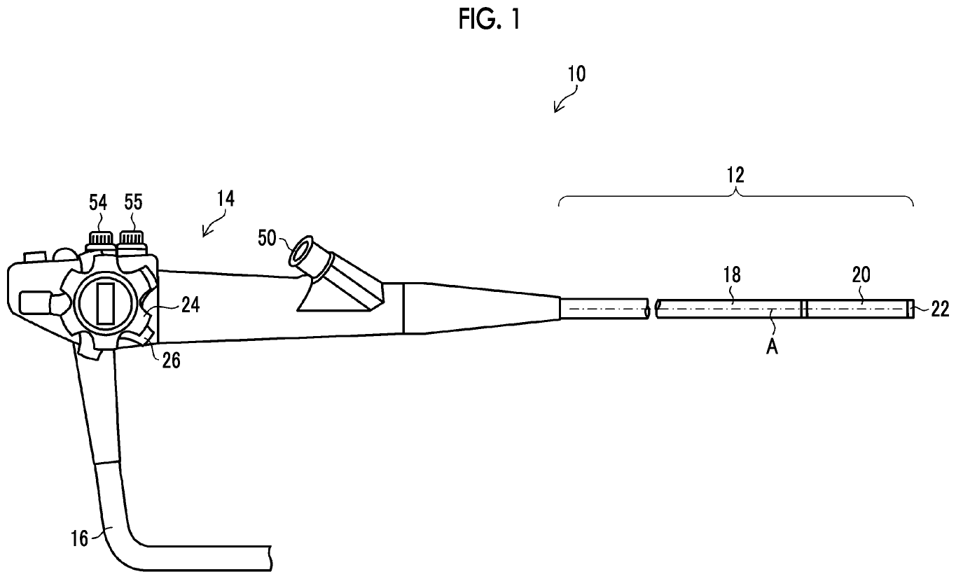

FIG. 1 is an overall view of an endoscope 10 according to the embodiment of the present invention.

As shown in FIG. 1, the endoscope 10 comprises an insertion part 12 inserted into a subject, an operation part 14 provided at a proximal end of the insertion part 12, and a universal cable 16 that connects the endoscope 10 to system configuration devices, such as a light source device, a processor device, and an air/water supply device (all not shown).

The insertion part 12 has a distal end, the proximal end, and a longitudinal axis A that is an axis of the insertion part 12, and comprises a soft portion 18, a bendable portion 20, and a distal end portion 22 in this order from the proximal end toward the distal end.

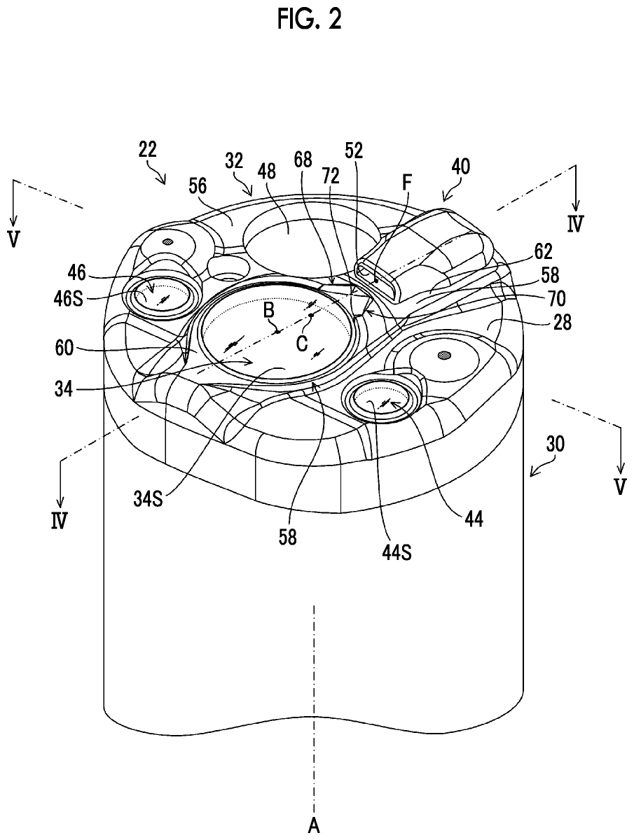

The soft portion 18 is flexible and is bendable in any direction along an insertion path of the insertion part 12. The bendable portion 20 is bent in each of an up-down direction and a left-right direction by the operation of angle knobs 24 and 26 rotatably provided in the operation part 14, and a direction that the distal end portion 22 faces can be changed to any direction. In addition, the distal end portion 22 has a distal end surface 28 (see FIG. 2) provided at the distal end of the insertion part 12.

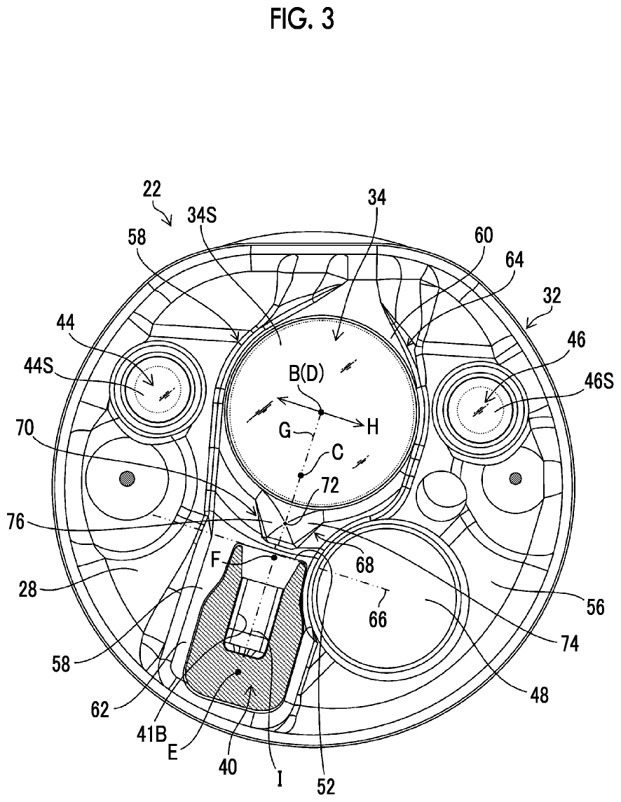

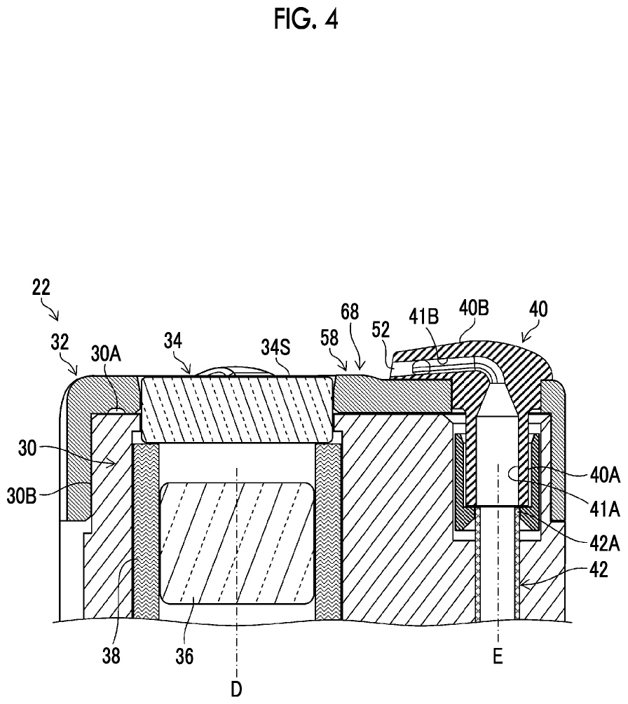

FIG. 2 is an enlarged perspective view of the distal end portion 22, and FIG. 3 is a front view of the distal end portion 22 as viewed from a front in a direction of the longitudinal axis A (see FIG. 1). Further, FIG. 4 is a cross-sectional view of the distal end portion 22 taken along line IV-IV of FIG. 2. In FIG. 3, a fluid jetting nozzle 40 is shown in a cross-sectional view in order to show a pipeline.

As shown in FIG. 4, the distal end portion 22 has a distal end portion body 30 that is made of a hard material, such as metal, and that holds various components disposed in the distal end portion 22, and a distal end cover 32 that is made of an insulating resin material and that covers a distal end surface 30A and a distal end outer peripheral surface 30B of the distal end portion body 30. FIG. 4 shows a lens barrel 38 that accommodates an optical system 36 and an observation window 34 constituting an observation portion, and a distal end part 42A of an air/water supply channel 42 that is connected to the fluid jetting nozzle (corresponding to the “nozzle”) 40, as components held by the distal end portion body 30 and the distal end cover 32.

The configuration of the distal end surface 28 of the distal end portion 22 will be described with reference to FIGS. 2 and 3. The distal end surface 28 is formed on a surface on a distal end side of the distal end cover 32. The distal end surface 28 is formed on the basis of a circular flat surface with a position intersecting with the longitudinal axis A as a center C of the distal end surface 28. In the following description, a “front side” indicates a distal end side of the insertion part 12 in the longitudinal axis A direction.

The distal end surface 28 comprises a forceps port forming surface portion 56 in which a forceps port 48 is formed. In addition, illumination windows 44 and 46 are provided on the same surface as the forceps port forming surface portion 56. The distal end surface 28 comprises a protruding surface portion 58 that protrudes to the front side with respect to the forceps port forming surface portion 56. The protruding surface portion 58 has an observation window-disposed surface portion 60 and a nozzle-disposed surface portion 62, and the observation window 34 is disposed on the observation window-disposed surface portion 60, and the fluid jetting nozzle 40 is disposed on the nozzle-disposed surface portion 62. The observation window-disposed surface portion 60 is provided so as to protrude to the front side with respect to the nozzle-disposed surface portion 62.

The forceps port 48 communicates with a forceps introduction port 50 of the operation part 14 via a forceps channel (not shown) inserted into the insertion part 12 (see FIG. 1). Therefore, a treatment tool introduced from the forceps introduction port 50 is led out of the forceps port 48 via the above-described forceps channel.

In addition, a suction channel (not shown) is connected to the above-described forceps channel, and a suction operation from the forceps port 48 is performed via the suction channel by the operation of a suction button 54 of the operation part 14 (see FIG. 1).

The illumination windows 44 and 46 are constituent elements of an illumination portion for illuminating a site to be observed, and irradiate the site to be observed with illumination light sent from the above-described light source device.

Circular surfaces 44S and 46S of the illumination windows 44 and 46 are each formed in, for example, a flat surface and are disposed perpendicular to the longitudinal axis A. Further, the centers of the surfaces 44S and 46S are each disposed at a position biased toward a peripheral edge of the distal end surface 28 with respect to the center C of the distal end surface 28 and are disposed at positions facing each other with a center B of a surface 34S of the observation window 34 interposed therebetween.

The observation window 34 is a constituent element of the observation portion for acquiring an image of the site to be observed and allows a solid-state imaging element (not shown) to capture subject light from the site to be observed via the optical system 36 shown in FIG. 4. The image captured by the observation portion is sent to the above-described processor device, as an image signal.

The circular surface 34S of the observation window 34 is formed in, for example, a flat surface and is disposed perpendicular to an optical axis D of the observation portion. Further, the center B of the surface 34S is disposed at a position biased toward the peripheral edge of the distal end surface 28 with respect to the center C of the distal end surface 28. The optical axis D is substantially parallel to the longitudinal axis A, and the center B is located on the optical axis D.

As shown in FIG. 4, the fluid jetting nozzle 40 has a proximal end part 40A and a distal end part 40B, and a shape including the proximal end part 40A and the distal end part 40B is formed in an L shape.

The proximal end part 40A constitutes a connecting part connected to the distal end part 42A of the air/water supply channel 42, and is connected to the above-described air/water supply device via the air/water supply channel 42. In addition, a pipeline 41A of the proximal end part 40A has a cross-section perpendicular to an axis of the pipeline 41A and formed in a circular shape, and a center E of the circular shape is disposed at a position biased toward the peripheral edge of the distal end surface 28 with respect to the center C (see FIG. 3) of the distal end surface 28 and is disposed at a position close to the illumination window 44, among the illumination windows 44 and 46, in order to avoid interference with the forceps port 48.

A pipeline 41B of the distal end part 40B has a cross-section perpendicular to an axis of the pipeline 41B and formed in a rectangular shape, and a jet port 52 that is open toward the observation window 34 is formed at a distal end of the pipeline 41B. The jet port 52 is formed as an opening having the same rectangular shape as the cross-sectional shape of the pipeline 41B.

In the fluid jetting nozzle 40 configured as described above, in a case where a leak hole (not shown) formed in an air/water supply button 55 of the operation part 14 (see FIG. 1) is closed with a finger, a gas from the air/water supply device is jetted from the jet port 52 toward the surface 34S of the observation window 34. Then, in a case where the air/water supply button 55 is pushed with the finger that has closed the above-described leak hole, a cleaning liquid from the air/water supply device is jetted from the jet port 52 toward the surface 34S of the observation window 34. As a procedure of cleaning the observation window 34, for example, a cleaning liquid is jetted from the jet port 52 to remove an attachment such as blood or body fluids adhering to the surface 34S of the observation window 34, and then a gas is jetted from the jet port 52 to remove the cleaning liquid remaining on the surface 34S or the like of the observation window 34.

Further, as shown in FIG. 3, in a case where a reference line 66 is assumed to be on an extension line in which the jet port 52 extends in a direction perpendicular to an axis I of the distal end part 40B of the fluid jetting nozzle 40 when the distal end surface 28 is viewed from the distal end side of the endoscope 10, the fluid jetting nozzle 40 and the forceps port 48 are located such that at least a part of the forceps port 48 is provided on the observation window 34 side with respect to the reference line 66. The forceps port 48 is provided close to the fluid jetting nozzle 40. In the present embodiment, the fluid jetting nozzle 40 is disposed on the protruding surface portion 58, and the forceps port 48 is provided close to the protruding surface portion 58.

(Configuration of Fluid Guide Portion)

Next, a configuration related to a flow passage of a fluid jetted from the jet port 52 will be described in detail.

As shown in FIGS. 2 to 4, the distal end surface 28 is provided with a fluid guide portion 68 that guides the fluid jetted from the jet port 52 to the observation window 34 or the like. The fluid guide portion 68 is provided between the fluid jetting nozzle 40 and the observation window 34 on the extension in which a rectangular opening region of the jet port 52 extends in a fluid jetting direction.

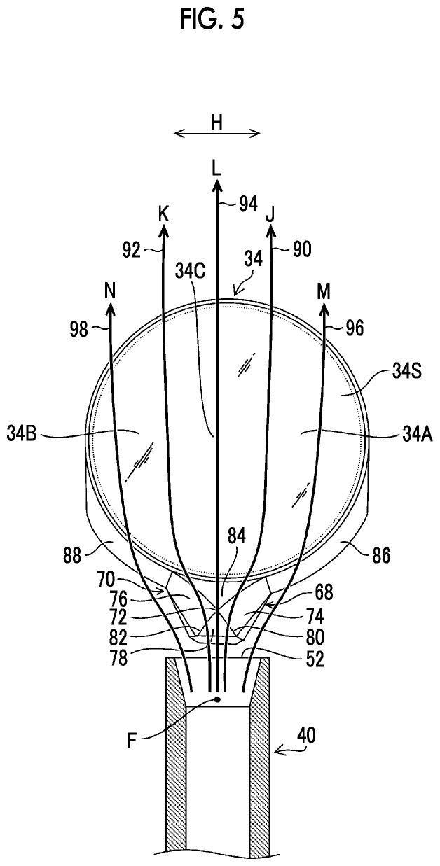

FIG. 5 is a plan view showing a configuration of the fluid guide portion and a fluid route of a fluid guided by the fluid guide portion. The fluid guide portion 68 includes a ridge portion 70 that rises up from the distal end surface 28 to the distal end side in the longitudinal axis A direction.

As shown in FIGS. 3 and 5, the ridge portion 70 has an apex portion 72 formed on a distal end side of the ridge portion 70 in the longitudinal axis A direction. In addition, the ridge portion 70 has a pair of first guide surfaces 74 and 76. The pair of first guide surfaces 74 and 76 are formed on both sides of the apex portion 72 in an arrow H direction orthogonal to a straight line G interconnecting a center F of the opening region of the jet port 52 and the center B of the observation window 34. The pair of first guide surfaces 74 and 76 function as guide surfaces that guide a part of the fluid jetted from the jet port 52 to both-side parts 34A and 34B of the observation window 34 in the arrow H direction, on the surface 34S of the observation window 34. FIG. 3 shows, as an example, a configuration in which the center C is located on the straight line G, but the present invention is not limited thereto, and a configuration may be employed in which the center C deviates from the straight line G.

As an example, the pair of first guide surfaces 74 and 76 are each formed of a slope that includes a component obliquely intersecting with the longitudinal axis A and that expands from the straight line G in the arrow H direction, from the fluid jetting nozzle 40 toward the observation window 34. The above-described slope is an example and may be formed of a surface having another shape as long as the fluid can be guided to the both-side parts 34A and 34B of the observation window 34.

In addition, the ridge portion 70 has a second guide surface 78. The second guide surface 78 is provided between the fluid jetting nozzle 40 and the apex portion 72 and functions as a guide surface that guides a part of the fluid jetted from the jet port 52 to a central part 34C of the observation window 34 via the apex portion 72.

As an example, the second guide surface 78 is formed of a slope inclined to the distal end side in the longitudinal axis A direction, from the fluid jetting nozzle 40 toward the observation window 34. The above-described slope is an example and may be formed of a surface having another shape as long as the fluid can be guided to the central part 34C of the observation window 34.

Further, the pair of first guide surfaces 74 and 76 and the second guide surface 78 are consecutively connected to each other via curved ridge line portions 80 and 82. Further, each of the pair of first guide surfaces 74 and 76 and the second guide surface 78 is formed of a surface including a streamlined curved surface.

Next, the action of the fluid guide portion 68 provided with the first guide surfaces 74 and 76 and the second guide surface 78 will be described.

As shown in FIG. 5, a part of the fluid jetted from the jet port 52 of the fluid jetting nozzle 40, specifically a fluid jetted from a position separated from the center F of the jet port 52 in the arrow H direction, flows from the second guide surface 78 to the pair of first guide surfaces 74 and 76 by way of the ridge line portions 80 and 82, as shown by first fluid routes 90 and 92 indicated by arrows J and K. Then, the fluid is guided to the both-side parts 34A and 34B of the observation window 34 by the pair of first guide surfaces 74 and 76 to flow through the both-side parts 34A and 34B. As a result, the both-side parts 34A and 34B of the observation window 34 are cleaned by the fluids flowing along the first fluid routes 90 and 92.

As described above, in the distal end surface 28 of the insertion part 12, since a configuration is employed in which the fluid guide portion 68 formed of the ridge portion 70 is provided between the fluid jetting nozzle 40 and the observation window 34, and a part of the fluid jetted from the jet port 52 is guided to the both-side parts 34A and 34B of the observation window 34 by the pair of first guide surfaces 74 and 76, which are formed on both sides of the apex portion 72, the cleanability of the both-side parts 34A and 34B of the observation window 34 can be improved.

In addition, since the pair of first guide surfaces 74 and 76 are each formed of a slope that includes a component obliquely intersecting with the longitudinal axis A and that expands in the arrow H direction, from the fluid jetting nozzle 40 toward the observation window 34, the fluid can be guided evenly toward the both-side parts 34A and 34B of the observation window 34.

Meanwhile, the fluids jetted from the center F of the jet port 52 and from the vicinity thereof are guided to the central part 34C of the observation window 34 via the apex portion 72 by the second guide surface 78 to flow through the central part 34C, as shown by a second fluid route 94 indicated by an arrow L. As a result, the central part 34C of the observation window 34 is cleaned by the fluid flowing along the second fluid route 94.

In addition, since the second guide surface 78 of the embodiment is formed of a slope inclined to the distal end side in the longitudinal axis A direction, from the fluid jetting nozzle 40 toward the observation window 34, the fluid can be guided evenly to the central part 34C of the observation window 34.

Further, since the pair of first guide surfaces 74 and 76 and the second guide surface 78 are consecutively connected to each other via the curved ridge line portions 80 and 82, the fluids flowing along the first fluid routes 90 and 92 can flow evenly from the second guide surface 78 toward the pair of first guide surfaces 74 and 76 via the ridge line portions 80 and 82. As a result, the fluid can be effectively guided toward the both-side parts 34A and 34B of the observation window 34.

Further, since the pair of first guide surfaces 74 and 76 each include the same streamlined curved surface, the fluid can be guided evenly from the pair of first guide surfaces 74 and 76 toward the both-side parts 34A and 34B of the observation window 34. Further, since the second guide surface 78 also includes a streamlined curved surface, the fluid can be guided evenly from the second guide surface 78 toward the central part 34C of the observation window 34. With the configuration in which the first guide surfaces 74 and 76 and the second guide surface 78 each include such a streamlined surface, most of the fluid jetted from the jet port 52 can be effectively used to clean the observation window 34 because the fluid can be restrained from scattering even in a case where the fluid forcefully collides with the pair of first guide surfaces 74 and 76 and the second guide surface 78.

The above-described streamlined surface indicates, for example, a curved surface that evenly expands from the distal end surface 28 toward the distal end side of the longitudinal axis A. In addition to the aspect in which the first guide surfaces 74 and 76 and the second guide surface 78 are each formed only of the above-described streamlined surface, a surface in which a streamlined surface and a flat surface are consecutively connected to each other may be used as long as the fluid can be guided evenly to the observation window 34.

As a preferred aspect, the fluid guide portion 68 of the present embodiment further comprises the following configurations.

The fluid guide portion 68 comprises a flat third guide surface 84 in order to allow the fluid that has passed through the second guide surface 78 to flow smoothly toward the observation window 34. The third guide surface 84 is provided, so that the fluid directed from the second guide surface 78 toward the central part 34C of the observation window 34 via the apex portion 72 can be guided evenly to the central part 34C of the observation window 34 by the third guide surface 84.

In addition, the fluid guide portion 68 comprises fourth guide surfaces 86 and 88 in order to guide a fluid deviated from the pair of first guide surfaces 74 and 76, among the fluids jetted from the jet port 52, to the both-side parts 34A and 34B of the observation window 34.

The fourth guide surfaces 86 and 88 are provided in the fluid guide portion 68, so that the fluid deviated from the pair of first guide surfaces 74 and 76 in the arrow H direction, among the fluids jetted from the jet port 52, is guided to the both-side parts 34A and 34B of the observation window 34 by the fourth guide surfaces 86 and 88 to flow through the both-side parts 34A and 34B, as shown by third fluid routes 96 and 98 indicated by arrows M and N. As a result, the both-side parts 34A and 34B can be cleaned together with the fluids flowing along the first fluid routes 90 and 92 indicated by the arrows J and K, so that the cleanability of the both-side parts 34A and 34B is further improved.

(Surface Characteristics of Distal End Surface)

Next, the surface characteristics of the distal end surface 28 will be described. In the present embodiment, in order to prevent a liquid remaining on the distal end surface 28 and a liquid remaining in the forceps port 48 because of incomplete suction from moving to the observation window 34 because of the gas jetted from the fluid jetting nozzle, water repellency and hydrophilicity are imparted to the distal end surface 28.

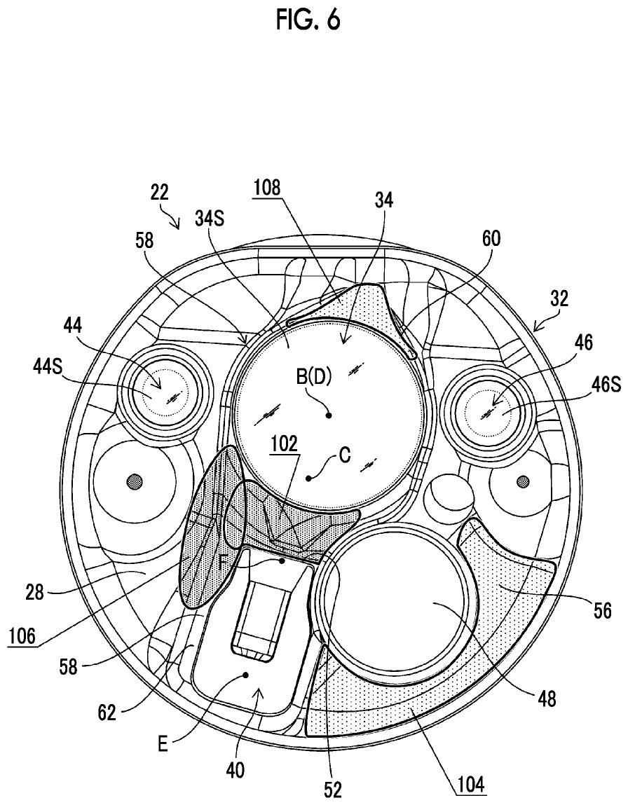

FIG. 6 is a view illustrating a water-repellent region and a hydrophilic region of the distal end surface 28. As shown in FIG. 6, in the distal end surface 28 of the present embodiment, a nozzle facing region 102 between the fluid jetting nozzle 40 and the observation window 34 is set as the water-repellent region. In addition, a forceps port adjacent region 104 provided on a side opposite to the nozzle facing region 102 with the forceps port 48 interposed therebetween is set as the hydrophilic region.

Further, the distal end surface 28 has, as the water-repellent region, a nozzle peripheral region 106 provided on a side opposite to the forceps port 48 with the nozzle facing region 102 interposed therebetween.

Further, the distal end surface 28 has, as the hydrophilic region, a fluid discharge region 108 provided on a side opposite to the nozzle facing region 102 with the observation window 34 interposed therebetween.

The water-repellent region can be formed by, for example, setting a surface roughness Ra of the distal end surface 28 to be smaller than 0.4. The water-repellent region can be formed by preferably setting the surface roughness Ra to be smaller than 0.2. In addition, the hydrophilic region can be formed by, for example, setting the surface roughness Ra to be larger than 0.4. In order to form the water-repellent region and the hydrophilic region by using the surface roughness, the entire region is cut so as to be the hydrophilic region (for example, Ra=0.4) through a mold polishing step, and then regions (the nozzle facing region 102 and the nozzle peripheral region 106) where water repellency is desired, in the cut surface, are polished (for example, polished such that Ra=0.2), whereby the water-repellent region and the hydrophilic region can be efficiently formed.

Further, as a contact angle with respect to the distal end surface 28, a contact angle with respect to the water-repellent region is preferably 80° or more. Further, a contact angle with respect to the hydrophilic region is preferably 70° or less. As the contact angle, a value measured using a “wettability evaluation device LSE-ME1 (contact angle meter)” manufactured by DAICO MFG CO., Ltd. can be used.

The water-repellent region can also be formed by applying a water-repellent coating, in addition to the above-described forming method using the surface roughness. As the water-repellent coating, a coating agent, such as a fluorine-based resin or a silicon-based resin, can be used, and the water-repellent region can be formed by applying and curing these coating agents.

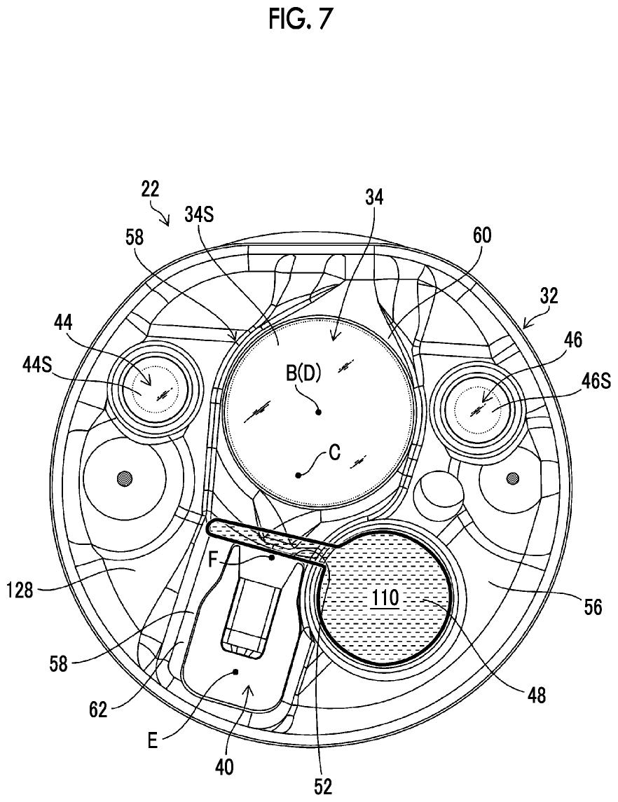

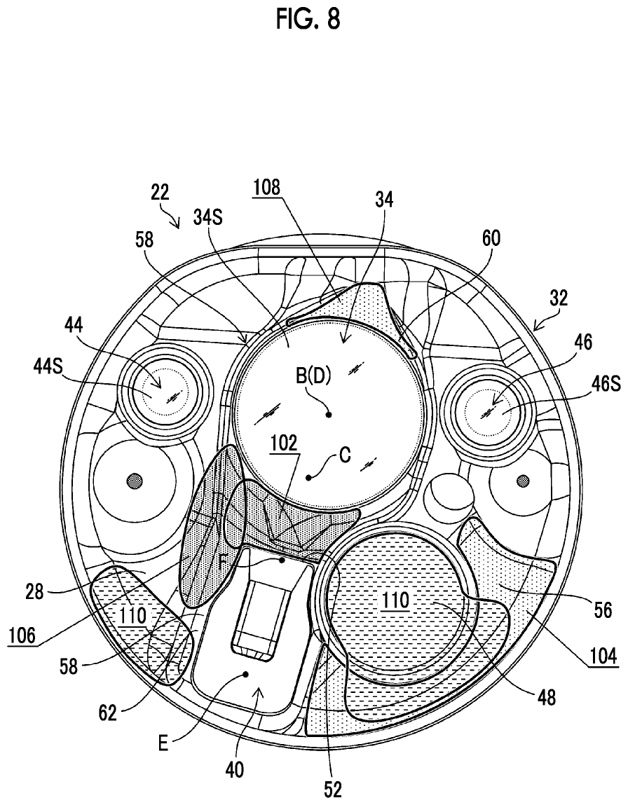

Next, the effect of providing the water-repellent region and the hydrophilic region on the distal end surface 28 will be described. FIG. 7 is a view illustrating a flow of a liquid on a distal end surface of an endoscope of a comparative example. FIG. 8 is a view illustrating a flow of a liquid on the distal end surface of the endoscope of the present embodiment.

A distal end surface 128 of the endoscope of the comparative example is a distal end surface that does not have the water-repellent region and the hydrophilic region on the distal end surface. In this case, as shown in FIG. 7, a liquid 110 remaining in the forceps port 48 is drawn out by surface tension between an end part of the forceps port 48 and a side surface of the protruding surface portion 58. Further, the liquid 110 is drawn out by the unevenness of the fluid jetting nozzle 40, the fluid guide portion 68, and the like provided on the distal end surface 128. In this state, in a case where a gas is jetted from the fluid jetting nozzle 40, the liquid 110 drawn out between the observation window 34 and the fluid jetting nozzle 40 is sent to the observation window 34. Further, since the liquid 110 remaining in the forceps port 48 is continuously drawn out by the gas jetted from the fluid jetting nozzle 40, the gas jetted from the fluid jetting nozzle 40 and the liquid 110 continuously move onto the observation window 34 in a mixed state. For that reason, the liquid may be reflected in the observation image, or the liquid remaining in the observation window 34 may not be removed, in many cases.

With the distal end surface 28 of the present embodiment, as shown in FIG. 8, the nozzle facing region 102 is made water-repellent, so that it is possible to prevent the liquid 110 remaining in the forceps port 48 because of incomplete suction from flowing into the region between the fluid jetting nozzle 40 and the observation window 34. In addition, the forceps port adjacent region 104 is made hydrophilic, so that the liquid 110 remaining in the forceps port 48 can be guided to the forceps port adjacent region 104. Liquids, such as residual water derived from the liquid 110 and a body fluid, are prevented from remaining in the nozzle facing region 102, so that it is possible to prevent the liquid 110 remaining in the forceps port 48 from being continuously drawn out and from moving to the observation window 34 side because of the gas jetted from the fluid jetting nozzle 40.

Further, the observation window 34 and the fluid jetting nozzle 40 are disposed on the protruding surface portion 58. In a case where the forceps port 48 and the protruding surface portion 58 are disposed close to each other, the liquid 110 remaining in the forceps port 48 is likely to be in a connected state between the forceps port 48 and the protruding surface portion 58 because of the surface tension. According to the present embodiment, since it is possible to prevent the liquid 110 from flowing into a region between the observation window 34 and the fluid jetting nozzle 40 by making the nozzle facing region 102 water-repellent, it is possible to prevent the liquid 110 from moving to the observation window 34 because of the gas jetted from the fluid jetting nozzle 40.

Further, the protruding surface portion 58 is provided with the nozzle-disposed surface portion 62 and the observation window-disposed surface portion 60 protruding to the front side with respect to the nozzle-disposed surface portion 62. Further, the fluid guide portion 68 is provided between the observation window 34 and the fluid jetting nozzle 40. Therefore, the liquid 110 remaining in the forceps port 48 is easily drawn out to the region therebetween. According to the present embodiment, since it is possible to prevent the liquid from flowing into the region between the observation window 34 and the fluid jetting nozzle 40 by making the nozzle facing region 102 water-repellent, it is possible to prevent the liquid from moving to the observation window 34 because of the gas jetted from the fluid jetting nozzle 40.

The positional relationship on the front side between the observation window-disposed surface portion 60 and the nozzle-disposed surface portion 62 is not limited thereto. The observation window-disposed surface portion 60 and the nozzle-disposed surface portion 62 may be provided on the same surface in the protruding surface portion 58. Further, the observation window 34 and the fluid jetting nozzle 40 may be disposed on the same surface as the forceps port forming surface portion 56, or any one thereof may be disposed on the same surface as the forceps port forming surface portion 56. Even with these configurations, since it is possible to prevent the liquid 110 from flowing into the region (nozzle facing region 102) between the observation window 34 and the fluid jetting nozzle 40 by making the nozzle facing region 102 water-repellent and the forceps port adjacent region 104 hydrophilic, it is possible to prevent the liquid 110 remaining in the forceps port 48 from moving to the observation window 34 because of the gas jetted from the fluid jetting nozzle 40.

In addition, since it is possible to prevent the liquid 110 remaining in the forceps port 48 from flowing into the nozzle facing region 102, it is possible to prevent the liquid 110 from moving to the observation window 34 even in a case where the position of the forceps port 48 is disposed on the observation window 34 side with respect to the reference line 66 in which the jet port 52 extends, as shown in FIG. 3. As a result, the position of the forceps port 48 can be disposed on the distal end surface 28 without limitation, so that the diameter of the insertion part can be reduced.

In addition, the fluid guide portion 68 is provided on the distal end surface of the endoscope 10 of the present embodiment, and the fluid jetted from the fluid jetting nozzle 40 effectively flows to both end parts of the observation window 34. At that time, as shown in FIGS. 2 and 3, in a case where the forceps port 48 is disposed adjacent to the fluid jetting nozzle 40, the gas jetted from the fluid jetting nozzle 40 is jetted to the vicinity of the forceps port 48. Therefore, in a case where the liquid sucked through the forceps port 48 remains in the forceps port 48, the liquid is drawn out by the gas jetted from the fluid jetting nozzle 40 and easily flows to the observation window 34. The forceps port adjacent region 104 is made hydrophilic, so that the liquid remaining in the forceps port 48 can be guided to the region on the opposite side with the forceps port 48 interposed therebetween, with respect to the path of the gas jetted from the fluid jetting nozzle 40. As a result, it is possible to prevent the liquid 110 remaining in the forceps port 48 from moving to the observation window 34 because of the gas jetted from the fluid jetting nozzle 40.

Further, the nozzle peripheral region 106 is made water-repellent, so that it is possible to prevent the liquid 110 remaining in the forceps port 48 from moving to the vicinity of the fluid jetting nozzle 40 provided on the side opposite to the forceps port 48 across the nozzle facing region 102, which is water-repellent. Furthermore, it is possible to prevent the liquid remaining on the distal end surface 28 from staying in the vicinity of the fluid jetting nozzle 40. Accordingly, it is possible to prevent the liquid from staying even in the vicinity of the fluid jetting nozzle 40 provided on the side opposite to the forceps port 48, and it is possible to prevent the liquid from moving to the observation window 34 because of the gas jetted from the fluid jetting nozzle 40.

In addition, the fluid discharge region 108 is made hydrophilic, so that the liquid present on the observation window 34 can be easily discharged from the fluid discharge region 108. As a result, the liquid present on the observation window 34 can be easily discharged by the gas jetted from the fluid jetting nozzle 40.

Although the nozzle facing region 102 is made water-repellent in FIG. 6, the entire protruding surface portion 58 may be made water-repellent. It is possible to prevent the liquid 110 remaining in the forceps port 48 from being connected to the protruding surface portion 58 by making the protruding surface portion 58 water-repellent because the fluid jetting nozzle 40 and the observation window 34 are disposed on the protruding surface portion 58. As a result, it is possible to prevent the liquid 110 from being drawn out onto the protruding surface portion 58 by the gas jetted from the fluid jetting nozzle 40, and it is possible to prevent the liquid 110 from moving to the observation window 34.

### EXPLANATION OF REFERENCES

10: endoscope
12: insertion part
14: operation part
16: universal cable
18: soft portion
20: bendable portion
22: distal end portion
24, 26: angle knob
28, 128: distal end surface
30: distal end portion body
30A: distal end surface of distal end portion body
30B: distal end outer peripheral surface of distal end portion body
32: distal end cover
34: observation window
34A: both-side part
34B: both-side part
34C: central part
34S: surface of observation window
36: optical system
38: lens barrel
40: fluid jetting nozzle
40A: proximal end part
40B: distal end part
41A: pipeline of proximal end part
41B: pipeline of distal end part
42: air/water supply channel
42A: distal end part of air/water supply channel
44, 46: illumination window
44S, 46S: surface of illumination window
48: forceps port
50: forceps introduction port
52: jet port
54: suction button
55: air/water supply button
56: forceps port forming surface portion
58: entire protruding surface portion
60: observation window-disposed surface portion
62: nozzle-disposed surface portion
66: reference line
68: fluid guide portion
70: ridge portion
72: apex portion
74, 76: first guide surface
78: second guide surface
80, 82: ridge line portion
84: third guide surface
86, 88: fourth guide surface
90, 92: first fluid route
94: second fluid route
96, 98: third fluid route
102: nozzle facing region
104: forceps port adjacent region
106: nozzle peripheral region
108: fluid discharge region
110: liquid
A: longitudinal axis
B: center of surface of observation window
C: center of distal end surface
D: optical axis of observation portion
E: center of proximal end part of fluid jetting nozzle
F: center of opening region of jet port
G: straight line interconnecting center of opening region of jet port and center of
observation window
I: axis of distal end part of fluid jetting nozzle

What is claimed is:

1. An endoscope comprising:
an insertion part having a distal end surface provided on a distal end side to be inserted into a subject;
an observation window that is provided in the distal end surface and that is used to observe an inside of the subject;
a nozzle that is provided in the distal end surface and that jets a fluid toward the observation window; and
a forceps port that is provided in the distal end surface and that is used to lead out a treatment tool or to suck the fluid,
wherein the distal end surface has: water repellency in a nozzle facing region which is provided between the nozzle and the observation window and is adjacent to a part of an opening end part of the forceps port; and hydrophilicity in a forceps port adjacent region which is provided on a side opposite to the nozzle facing region with the forceps port interposed therebetween and is adjacent to other part of the opening end part of the forceps port.

2. The endoscope according to claim 1,
wherein the distal end surface has a forceps port forming surface portion in which the forceps port is formed, and a protruding surface portion formed so as to protrude toward a front side, which is the distal end side, with respect to the forceps port forming surface portion, and
at least one of the observation window or the nozzle is disposed on the protruding surface portion.

3. The endoscope according to claim 2,
wherein the protruding surface portion has an observation window-disposed surface portion in which the observation window is disposed, and a nozzle-disposed surface portion in which the nozzle is disposed, and
the observation window-disposed surface portion protrudes to the front side with respect to the nozzle-disposed surface portion.

4. The endoscope according to claim 2,
wherein the protruding surface portion has water repellency.

5. The endoscope according to claim 1,
wherein the forceps port is disposed at a position close to the nozzle facing region.

6. The endoscope according to claim 1,
wherein the distal end surface has water repellency in a nozzle peripheral region provided on a side opposite to the forceps port with the nozzle facing region interposed therebetween.

7. The endoscope according to claim 1,
wherein the nozzle has a jet port for jetting the fluid, and
at least a part of the forceps port is provided on an observation window side with respect to a reference line, which is an extension line of the jet port.

8. The endoscope according to claim 1,
wherein the distal end surface has hydrophilicity in a fluid discharge region provided on a side opposite to the nozzle facing region with the observation window interposed therebetween.
